(19) United States
(12) Patent Application Publication
(10) Pub. No.: US 2017/0106088 A1
(43) Pub. Date: Apr. 20, 2017

(54) MAGNETIC DIRECTED ALIGNMENT OF STEM CELL SCAFFOLDS FOR REGENERATION

(71) Applicants: UNIVERSITY OF HOUSTON SYSTEM, Houston, TX (US); BOARD OF REGENTS OF THE UNIVERSITY OF TEXAS SYSTEM, Austin, TX (US)

(72) Inventors: Dong LIU, Houston, TX (US); Qilin CAO, Pearland, TX (US)

(73) Assignees: UNIVERSITY OF HOUSTON SYSTEM, Houston, TX (US); BOARD OF REGENTS OF THE UNIVERSITY OF TEXAS SYSTEM, Austin, TX (US)

(21) Appl. No.: 15/315,247

(22) PCT Filed: Jun. 3, 2015
(86) PCT No.: PCT/US2015/033987

§ 371 Date: Nov. 30, 2016

Related U.S. Application Data
(60) Provisional application No. 62/007,197, filed on Jun. 3, 2014.

(51) Int. Cl. A61K 41/00; A61K 35/33; A61K 35/15; A61K 47/02; C12N 5/00; A61K 9/107; A61K 47/34; A61K 38/18; C12N 5/0797; C12N 5/077; A61K 35/30; A61K 47/36

(52) U.S. Cl. CPC A61K 41/00; A61K 35/30; A61K 35/33; A61K 35/15; A61K 47/02; A61K 47/36; A61K 9/107; A61K 47/34; A61K 38/185; C12N 5/0623; C12N 5/0656; C12N 5/0062; C12N 2529/00

(57) ABSTRACT

The present disclosure relates to liposomal delivery vehicles comprising magnetic particles and their use in modifying stem cells for delivery to target sites such as neuronal tissues.

This application claims benefit of priority to U.S. Provisional Application Ser. No. 62/007,197, filed Jun. 3, 2014, the entire contents of which are hereby incorporated by reference.

This invention was made with government support under Grant Award #1134119 awarded by the National Science Foundation. The government has certain rights in the invention.

### BACKGROUND

A. Field

In one aspect, the present disclosure relates generally to the fields of medicine and neurobiology. More particularly, it concerns the liposomal delivery vehicles comprising magnetic particles and their use in modifying stem cells for delivery to target sites such as neuronal tissues.

B. Description of Related Art

As a primary nervous system disorder, SCI causes severe and chronic debilitation or even permanent disability to millions of people in the US and worldwide. SCI affects the young people (aged from 16 to 30), mainly due to motor vehicle accidents or battlefield injuries, as well as senior citizens (aged 70 and above) due to aging-related stenosis or bone quality change. A person suffering from SCI may incur between $500,000 and $3.1 million in life time medical expenses. The economic costs for SCI are well beyond $10 billion per year in the US alone [1]. Besides the heavy familial, social and economic burden, the patients' physical and emotional suffering is immense.

Despite the extensive research efforts and improvement in the rehabilitation approaches, unfortunately, SCI continues to be a significant cause of disability and mortality. Indeed, effective treatments are currently not available for the spinal cord injury (SCI)-triggered sensory and motor impairments, primarily due to the lack of successful strategies being optimally developed to promote long distance axonal regeneration. Unfortunately, most axons in the adult mammalian central nervous system (CNS) fail to regenerate after injury. This has been attributed not only to the intrinsic indolence of the mature neurons, but also to the non-permissive environment encountered by the injured axons. Therefore, novel therapeutic approaches to modify the inhibitory environment to promote axonal regeneration are urgently needed.

### SUMMARY

Thus, in accordance with the present disclosure, there is provided a method of treating a cell comprising contacting the cell with a liposome comprising a plurality of magnetic iron particles. The cell may be a stem cell. The liposome may be a cationic liposome, such as one comprising TMAG, DOPE, and/or DLPC. The stem cell may be a neural stem cell. The liposome may further comprise a growth factor. The growth factor may be neurotrophin 3 or brain-derived neurotrophin factor. The magnetic particles may be iron oxide nanoparticles. The iron nanoparticles may be about 5 to about 20 nm in diameter. The liposome may be about 100 to about 500 nm in diameter. The iron magnetic particles are surface modified with a biocompatible organic molecule, such as dextran or a protein. The cell may be a fibroblast.

In another embodiment, there is provided a liposome comprising (a) a plurality of magnetic iron nanoparticles coated with a biocompatible organic polymer and (b) a growth factor. The growth factor may be a neurogenic growth factor. The liposome may be a cationic liposome, such as TMAG, DOPE, and/or DLPC.

In still a further embodiment, there is provided a method of forming a cell structure comprising applying a stationary magnetic field to a plurality of cells comprising liposomes comprising a plurality of magnetic iron particles. The cell structure may be a cell lattice or chain. The cell structure may be formed in a living subject. The cell structure may comprise neural stem cells, and may be located in a nerve site in said subject. The subject may suffer from a nerve deficit, such as one resulting from traumatic nerve injury, from a spinal cord injury, from a peripheral nerve injury, or a neurodegenerative disease, such as Parkinson's disease, Huntingtin's disease, amyotrophic lateral sclerosis, Alzheimer's disease, Friederich's ataxia, Lewy body disease, or spinal muscular dystrophy.

The liposome may be a cationic liposome, such as one comprising TMAG, DOPE, and/or DLPC. The stem cell may be a neural stem cell. The liposome may further comprise a growth factor. The growth factor may be neurotrophin 3 or brain-derived neurotrophin factor. The magnetic iron particles may be iron oxide nanoparticles. The magnetic iron nanoparticles may be about 5 to about 20 nm in diameter. The liposome may be about 100 to about 500 nm in diameter. The magnetic iron particles are surface modified with a biocompatible organic molecule, such as dextran or a protein. The cell may be a fibroblast.

As used herein the specification, “a” or “an” may mean one or more.

As used herein in the claim(s), when used in conjunction with the word “comprising,” the words “a” or “an” may mean one or more than one.

As used herein “another” may mean at least a second or more.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B—Current scaffolds fabricated for SCI repair [14,15].

FIGS. 2A-B—Magnetic field directed self-assembly of cells. (FIG. 2A) 2D fibrin gel scaffold created with a magnetically-oriented particle array [29]. (FIG. 2B) Tubular tissue fabricated using Mag-TE technique [30].

FIG. 3—Schematic of magnetoliposome (ML) that can function as combined magnetic labeling and drug delivery agents.

FIG. 4—Magnetically directed self-assembly of NSC chain lattices labeled with CMLs.

FIGS. 5A-C—Electrochemical synthesis of SPIONs. (FIG. 5A) Schematic of Fe₃O₄ SPION synthesis process. (FIG. 5B) TEM micrograph of SPIONs. (FIG. 5C) room temperature superparamagnetic response of SPIONs.

FIGS. 6A-B—Micrographs of synthesized liposomes. (FIG. 6A) CLs produced from reverse evaporation. (FIG. 6B) CMLs produced from invert emulsion.

FIGS. 7A-D. Optical micrographs of a confluent culture of Rat-2 fibroblast cells after 24-hour exposure to: (FIG. 7A) N(CH3)4OH-coated SPIONs, where the exposure destroys all trace of cell activity and membrane structure; (FIG. 7B) bare SPIONs, where the viability is reduced to less than 20% as determined by Trypan blue staining (dark cells), and (FIG. 7C) dextran-coated SPIONs, where confluent cell structure is maintained and viability is greater than 90%, comparable to the control experiment without nanoparticles are added. The 3-(4,5-dimethylthiazol-2-yl)-5-(3-carboxymethoxyphenyl)-2-(4-sulfophenyl)-2H-tetrazolium (MTS) assay is used to quantitatively determine the cytotoxicity of CMLs on human fibroblast cells with two initial cell concentrations, low (125,500 per mL) and high (223,000 per mL) (FIG. 7D). The slope of the MTS measurement represents the cellular decease rate, thus the results demonstrate that the viability of CML-labeled cells at all CML concentrations (0.5, 1.5 and 2.5 mg/mL) is almost identical to that of cells with no nanoparticle internalization.

FIGS. 8A-B—Directed self-assembly of micro/nanoparticles. (FIG. 8A) Polystyrene microparticles assemble into chain lattices under negative dielectrophoresis. (FIG. 8B) Nickel nanowires form continuous chains with magnetic field.

FIGS. 9A-C—Microphotographs of OPCs labeled with SPIONs. (FIG. 9A) Cells expanded in vitro in a culture flask, note the evidence of intracellular uptake of SPIONs. (FIG. 9B) Cells harvested by using trypsin are still alive after 24˜48 hours exposure to SPIONs. (FIG. 9C) cell alignment after the magnetic field is applied.

FIGS. 10A-I—Transplanted NSCs promote axonal regeneration after SCI. One month after C3 dorsal funiculus laceration, both rostral and caudal axons, labeled by NFM (FIG. 10A; left and right), fail to regenerate cross the injury site in control injured animals. The ascending DC axons, labeled by CTB, stop at the caudal side of injured spinal cord (FIG. 10B, right). No CTB+ DC axons are found in the spinal cord rostral to the injury site (FIG. 10B, left). Similarly, the corticospinal tracts, labeled by BDA, fail to cross the injury site (FIG. 10C). No BDA+ axons are found in the spinal cord caudal to the injury site (FIG. 10D). One month after transplantation into the injured spinal cord, grafted NSCs survive and fill the injury gap (FIG. 10E). They also migrate extensively into both rostral and caudal spinal cord (FIG. 10E). Pan-A+ axons from both rostral and caudal spinal cord regenerate across the injured site filled by grafted NSCs (FIG. 10F). CTB-labeled ascending DC axons grow across the injury site (FIGS. 10G-H, right line) and reach the spinal cord rostral to the injury site (FIG. 10F, left line). While many BDA-labeled CST axons stop at the rostral side of the injured spinal cord, some BDA-labeled CST axons regenerate across the injured gap filled with BrdU-labeled NSCs (FIG. 10I, between lines).

FIGS. 11A-C—Schematic of magnetic directed assembly of NSC chain lattices: (FIG. 11A) Induced moment in the NSC. (FIG. 11B) Formation of chain lattices. (FIG. 11C) Micrograph of aligned microparticles of a 10 μm diameter [27].

FIGS. 12A-F—Schematic of the kinetics of particle aggregation as the magnetic field strength increases.

FIG. 13—Magnetic levitation of Rat fibroblast cell lines. The magnetically labeled cells are successfully levitated and form a loosely connected aggregate after 5 hours' incubation. As time elapses, the 3D cell assembly becomes more concentrated: the micrographs taken after 48 hrs and 72 hrs incubation. After removing the magnetic field, the levitated cell structure retains the ability to proliferate after being incubated for another 40 hrs (Bottom panel, left to right).

FIG. 14—3D Alignment of Rat fibroblast cell line at low cell concentration (62,500 cells per mL). Center and right panels show instantaneous alignment at a magnetic field strength of 50 Gauss.

FIGS. 15A-E—3D Alignment of Rat fibroblast cell line at high cell concentration after 60 hr incubation at indicated magnification.

FIG. 16—3D Alignment and growth of Human lung adenocarcinoma epithelial cell line (A549) at various concentrations (6.25×10⁴, 1.25×10⁵, 2.5×10⁵, 5×10⁵, and 2.0×10⁶ cells/well.) after 72 hrs incubation.

FIG. 17—3D Alignment and growth of human neural stems cells after 48 hrs incubation. DAPI (4′,6-diamidino-2-phenylindole) nucleic acid staining method is used to explore the viability of the 3D NSC column structure.

### DESCRIPTION OF ILLUSTRATIVE EMBODIMENTS

Neural stem cells (NSCs) have shown great promise to promote axonal regeneration and functional recovery after SCI, likely an effective novel therapy. The present disclosure is designed to greatly advance neural stem cell-based therapy in clinical treatments of SCI, and potentially offer the ultimate cure for a large variety of patients who are suffering from SCI. The technology of using magnetically-labeled cells to construct injectable, alignable and bioactive scaffolds may also have far-reaching impact on neural tissue engineering and regenerative medicine in general.

These and other aspects of the disclosure are described in detail below.

### A. SPINAL CORD INJURY AND REPAIR

1. SCI

SCI is difficult to heal partially because the central nervous system (CNS) has very limited capacity to repair itself after an injury. Mature CNS neurons have an intrinsic indolence to regenerate. Previous experiments [2] showed that severed axons initially attempted to regenerate spontaneously, but quickly lost the ability to do so after failing to find their way in the right direction to re-establish the nerve connection. Additionally, the non-permissive environment encountered by the injured axons, which includes the molecules in myelin (the insulating sheath surrounding nerve fibers) carrying inhibitory factors and the scar that form after the injury, contributes to their inability to regenerate.

Accordingly, several repair strategies have been explored to promote axonal regeneration in the injured spinal cord, those including: 1) enhancing the intrinsic regenerative capacity of injured neurons [5-7]; 2) providing permissive neurotrophic factors [8, 9]; 3) transplantation of regeneration-permissive cells, such as Schwann cells, olfactory ensheathing glia or NSCs; and 4) removing the inhibitory factors in the injured spinal cord. Since the mechanism of action for each of these strategies is distinct, one would expect that combining multiple strategies would bring about greater axonal regeneration. The NSC-based cell therapy represents such a promising approach. Grafted NSCs and glial precursor cells (GPCs) have shown great potential to promote axonal regeneration after SCI. They can replace the lost neurons and oligodendrocytes to promote the remyelination of demyelinated and degenerated axons.

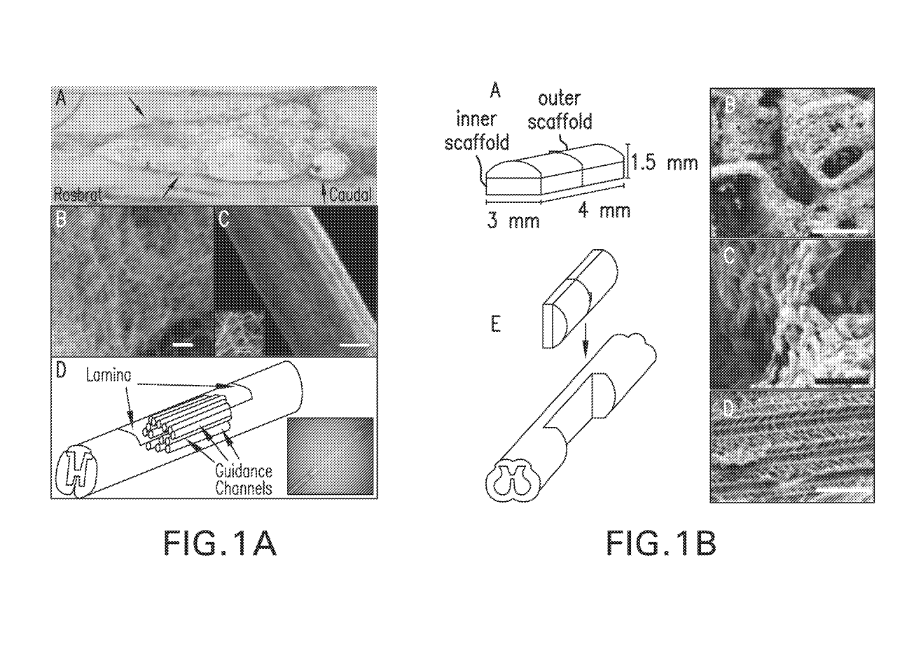

NSCs also constitutively secrete neurotrophic factors and other permissive substances to stimulate axonal growth across an injury gap [10, 11]. After transplantation into the transected spinal cord, the NSCs promote regeneration of the corticospinal tract across the injury site to reach the caudal spinal cord where the regenerating axons reform synapses. However, a challenge exists for the above NSC-based cell therapy, especially in chronic SCI—when the entire cross section of the spinal cord is replaced with scar tissue and/or fluid-filled cysts, the mechanical substrates that provide mechanical support for axonal regeneration and the three-dimensional cytoarchitectural information become permanently lost [12]. Fortunately, biological scaffolds seeded with NSCs can provide a solution, which not only offer structural support for the attachment of grafted NSCs in the injury site but also sustain a controlled biochemical microenvironment to induce the differentiation of NSCs to desired cell lineages. Current scaffolds for SCI are mostly made of polymer composites that are assembled in the form of porous biomatrix or guidance microconduits, as shown in FIGS. 1A-B [12-15]. While the bioscaffolds for SCI repair show promising attributes, such as mechanical compliance, biocompatibility and degradation, high porosity and functionalization with bioactive motifs, almost all of them have to be fabricated in vitro and then implanted into the injured spinal cord through invasive surgical operations.

An ideal scaffold for SCI repair is envisioned in the proposed research, which possesses the following attributes: (1) injectability: the constituent materials of the scaffold can be directly injected as aqueous suspensions into the injured spinal cord, and afterward the scaffold will establish itself spontaneously in vivo; (2) alignability: the formed scaffold will be aligned along a preferential direction, i.e., longitudinally in the spinal cord, to guide the regrowth of axons to bridge the injury gap; and (3) bioactivity: growth factors can be accommodated in the scaffold and be released in a controlled way to promote the survival of grafted NSCs and the axonal regeneration. Currently, there are no technologies capable of fabricating scaffolds with those attributes.

2. Stem Cells

Stem cells are undifferentiated biological cells that can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. There are three accessible sources of autologous adult stem cells in humans:
Bone marrow, which requires extraction by harvesting, that is, drilling into bone (typically the femur or iliac crest), adipose tissue (lipid cells), which requires extraction by liposuction, and blood, which requires extraction through apheresis, wherein blood is drawn from the donor (similar to a blood donation), and passed through a machine that extracts the stem cells and returns other portions of the blood to the donor. Stem cells can also be taken from umbilical cord blood just after birth. Of all stem cell types, autologous harvesting involves the least risk. By definition, autologous cells are obtained from one's own body, just as one may bank his or her own blood for elective surgical procedures.
Adult stem cells are frequently used in medical therapies, for example in bone marrow transplantation. Stem cells can now be artificially grown and transformed (differentiated) into specialized cell types with characteristics consistent with cells of various tissues such as muscles or nerves. Embryonic cell lines and autologous embryonic stem cells generated through therapeutic cloning have also been proposed as promising candidates for future therapies.
Neural stem cells (NSCs) are self-renewing, multipotent cells that generate the main phenotypes of the nervous system. Stem cells are characterized by their capability to differentiate into multiple cell types via exogenous stimuli from their environment. They undergo asymmetric cell division into two daughter cells, one non-specialized and one specialized. NSCs primarily differentiate into neurons, astrocytes, and oligodendrocytes.
   The main distinction between stem cells is that one is an adult stem cell which is limited in its ability to differentiate and one is an embryonic stem cell (ESC) that is pluripotent. ESCs are not limited to a particular cell fate; rather they have the capability to differentiate into any cell type. ESCs are derived from the inner cell mass of the blastocyst with the potential to self-replicate.

NSCs are considered adult stem cells because they are limited in their capability to differentiate. NSCs are generated throughout an adult's life via the process of neurogenesis. Since neurons do not divide within the central nervous system (CNS), NSCs can be differentiated to replace lost or injured neurons or in many cases even glial cells. NSCs are differentiated into new neurons within the SVZ of lateral ventricles, a remnant of the embryonic germinal neuroepithelium, as well as the dentate gyrus of the hippocampus. Epidermal growth factor (EGF) and fibroblast growth factor (FGF) are mitogens that promote neural progenitor and stem cell growth in vitro, though other factors synthesized by the neural progenitor and stem cell populations are also required for optimal growth. It is hypothesized that neurogenesis in the adult brain originates from NSCs. The origin and identity of NSCs in the adult brain remain to be defined.

Adult NSCs were first isolated from mouse striatum in the early 1990's. They are capable of forming multipotent neurospheres when cultured in vitro. Neurospheres can produce self-renewing and proliferating specialized cells. These neurospheres can differentiate to form the specified neurons, glial cells, and oligodendrocytes. In previous studies, cultured neurospheres have been transplanted into the brains of immunodeficient neonatal mice and have shown engraftment, proliferation, and neural differentiation.

NSCs are stimulated to begin differentiation via exogenous cues from the microenvironment, or stem cell niche. This capability of the NSCs to replace lost or damaged neural cells is called neurogenesis. Some neural cells are migrated from the SVZ along the rostral migratory stream which contains a marrow-like structure with ependymal cells and astrocytes when stimulated. The ependymal cells and astrocytes form glial tubes used by migrating neuroblasts. The astrocytes in the tubes provide support for the migrating cells as well as insulation from electrical and chemical signals released from surrounding cells. The astrocytes are the primary precursors for rapid cell amplification. The neuroblasts form tight chains and migrate towards the specified site of cell damage to repair or replace neural cells. One example is a neuroblast migrating towards the olfactory bulb to differentiate into periglomercular or granule neurons which have a radial migration pattern rather than a tangential one.

On the other hand, the dentate gyrus neural stem cells produce excitatory granule neurons which are involved in learning and memory. One example of learning and memory is pattern separation, a cognitive process used to distinguish similar inputs.

Cell death is a characteristic of acute CNS disorders as well as neurodegenerative disease. The loss of cells is amplified by the lack of regenerative abilities for cell replacement and repair in the CNS. One way to circumvent this is to use cell replacement therapy via regenerative NSCs. NSCs can be cultured in vitro as neurospheres. These neurospheres are composed of neural stem cells and progenitors (NSPCs) with growth factors such as EGF and FGF. The withdrawal of these growth factors activates differentiation into neurons, astrocytes, or oligodendrocytes which can be transplanted within the brain at the site of injury. The benefits of this therapeutic approach have been examined in Parkinson's disease, Huntington's disease, and multiple sclerosis. NSPCs induce neural repair via intrinsic properties of neuroprotection and immunomodulation. Some possible routes of transplantation include intracerebral transplantation and xenotransplantation.

An alternative therapeutic approach to the transplantation of NSPCs is the pharmacological activation of endogenous NSPCs (eNSPCs). Activated eNSPCs produce neurotrophic factors, several treatments that activate a pathway that involves the phosphorylation of STAT3 on the serine residue and subsequent elevation of Hes3 expression (STAT3-Ser/Hes3 Signaling Axis) oppose neuronal death and disease progression in models of neurological disorder.

Traumatic Brain Injury (TBI) can deform the brain tissue, leading to necrosis primary damage which can then cascade and activate secondary damage such as excitotoxicity, inflammation, ischemia, and the breakdown of the blood-brain-barrier. Damage can escalate and eventually lead to apoptosis or cell death. Current treatments focus on preventing further damage by stabilizing bleeding, decreasing intracranial pressure and inflammation, and inhibiting pro-apoptoic cascades. In order to repair TBI damage, an upcoming therapeutic option involves the use of NSCs derived from the embryonic periventricular egion. Stem cells can be cultured in a favorable 3-dimensional, low cytotoxic environment, e.g., a hydrogel, that will increase NSC survival when injected into TBI patients. The intracerebrally injected, primed NSCs have been seen to migrate to damaged tissue and differentiate into oligodendrocytes or neuronal cells that secreted neuroprotective factors.

### B. MAGNETIC NANOPARTICLES AND MAGNETOLIPOSOMES

1. SPIONs

Superparamagnetic iron oxide nanoparticles (SPIONs) with a diameter less than 20 nm exhibit strong magnetization only when they are exposed to an external magnetic field, otherwise they show no remnant magnetism [16]. SPIONs have several attributes for biomedical applications. If cells are labeled with SPIONs, the coupling between the magnetic field and SPIONs allows remote detection of the cellular components in a body, regardless of whether there are intervening structures [17]. SPIONs can also be surface-functionalized with specific biomolecules. Furthermore, SPIONs can form very stable colloidal suspensions. As a result, SPIONs have been used as effective imaging tracer tags and drug delivery vehicles, including the magnetic drug targeting (MDT), magnetofection and gene delivery (MF), magnetic resonance imaging (MRI) and magnetic fluid hyperthermia (MFH) [18-22].

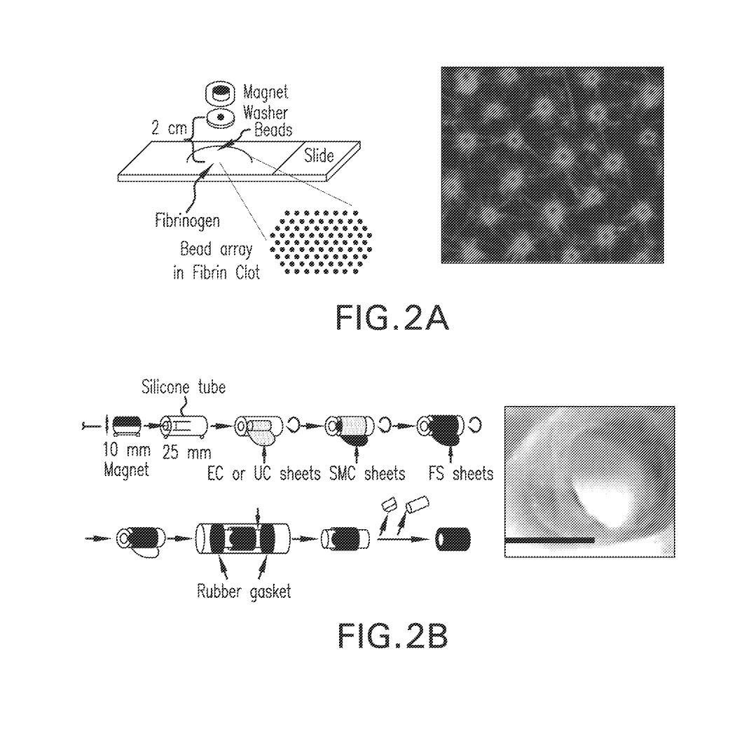

More recently, SPIONs have been used for cellular mechanical conditioning and scaffold fabrication in tissue engineering. In the first type of application, SPIONs attached to the integrin receptors on the cell surface can induce precisely controlled magnetic forces to target and activate individual mechanosensitive ion channels [23-25]. The second type of application hinges upon the fact that when guided by an external magnetic field, SPIONs can self-assemble into one-, two- or three-dimensional ordered supraparticle structures [26-28]. FIG. 2A-B shows two examples. In FIG. 2A, magnetic forces were used to position thrombin-coated microparticles in two-dimensional hexagonal arrays which direct the self-assembly of fibrin fibrils [29]. Fibrin matrices with defined nanoscale architecture were fabricated that support adhesion and spreading of human endothelial cells. As shown in FIG. 2B, tubular structures made of multilayered sheets of epithelial tissues were created using a “magnetic force-based tissue engineering” (Mag-TE) technique in which the SPION-labeled cells were manipulated and patterned with a magnet [30-33].

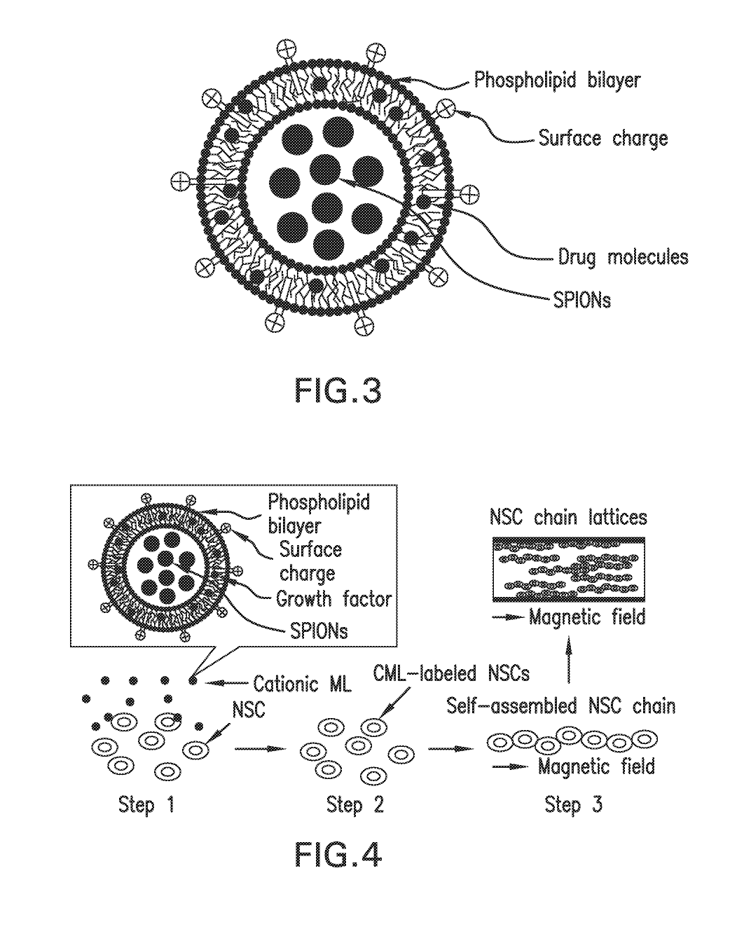

SPIONs in their native unmodified form are inefficient intracellular labels for stem cells and other mammalian cells [34-36]. They must be surface-modified to improve the biocompatibility and to increase functionality. SPIONs can be coated with synthetic or organic polymers (such as dextrans or proteins) and amphiphilic molecules (such as fatty acids or phospholipids) [37]. When a group of clustered SPIONs is encapsulated in the interior of a bilayer of amphiphilic phospholipids, magnetoliposomes (MLs) are formed [38, 39], as schematically shown in FIG. 3. When a magnetic field is applied, the individual magnetic dipole moments of SPIONs inside the MLs will lead synergistically to an enhanced cumulative dipole moment, making the MLs highly efficient for MRI tracking and magnetic-force-based manipulation. Since the phospholipids are natural substances, MLs have excellent biocompatibility. When the MLs are internalized by cells, the SPIONs will be shielded from degradation by the strongly chemisorbed phospholipid layer, and can persist during continuous cell proliferation. Another benefit of MLs comes from the wide variety of phospholipids that can be used to synthesize them and the ease with which their surfaces can be modified chemically by specific targeting ligands, both greatly increasing the cellular uptake efficiency and reducing cytotoxicity of the MLs. More importantly, hydrophilic and hydrophobic drugs can be hosted either within the lipid bilayer or in the inner aqueous cavity of the MLs, making them good drug delivery carriers [37]. Release of the drugs can be triggered and controlled by increasing the local temperature (below the limit of hyperthermia) via heating the nanoparticles with radio frequency (RF) electromagnetic fields [40].

In this project, cationic MLs (CMLs) will be synthesized with superior properties for cellular uptake and drug delivery. Since cellular membranes bear an overall negative charge, the positively charged CMLs can be easily internalized via electrostatic interaction. Inclusion of hydrophilic neurotrophin factors, such as neurotrophin 3 (NT3) and brain derived neurotrophin factor (BDNF), within the lipid bilayer can also be exploited.

SPIONs in their native unmodified form are inefficient intracellular labels for stem cells and other mammalian cells [34-36]. They must be surface-modified to improve the biocompatibility and to increase functionality. SPIONs can be coated with synthetic or organic polymers (such as dextrans or proteins) and amphiphilic molecules (such as fatty acids or phospholipids) [37]. When a group of clustered SPIONs is encapsulated in the interior of a bilayer of amphiphilic phospholipids, magnetoliposomes (MLs) are formed [38, 39], as schematically shown in FIG. 3. When a magnetic field is applied, the individual magnetic dipole moments of SPIONs inside the MLs will lead synergistically to an enhanced cumulative dipole moment, making the MLs highly efficient for MRI tracking and magnetic-force-based manipulation. Since the phospholipids are natural substances, MLs have excellent biocompatibility. When the MLs are internalized by cells, the SPIONs will be shielded from degradation by the strongly chemisorbed phospholipid layer, and can persist during continuous cell proliferation. Another benefit of MLs comes from the wide variety of phospholipids that can be used to synthesize them and the ease with which their surfaces can be modified chemically by specific targeting ligands, both greatly increasing the cellular uptake efficiency and reducing cytotoxicity of the MLs. More importantly, hydrophilic and hydrophobic drugs can be hosted either within the lipid bilayer or in the inner aqueous cavity of the MLs, making them good drug delivery carriers [37]. Release of the drugs can be triggered and controlled by increasing the local temperature (below the limit of hyperthermia) via heating the nanoparticles with radio frequency (RF) electromagnetic fields [40].

2. CMLs

Cationic magnetic liposomes (CMLs) will be synthesized with superior properties for cellular uptake and drug delivery. Since cellular membranes bear an overall negative charge, the positively charged CMLs can be easily internalized via electrostatic interaction.

3. Growth Factors

Hydrophilic neurotrophin factors, such as neurotrophin 3 (NT3) and brain derived neurotrophin factor (BDNF) may be included within the lipid bilayer can also be exploited.

### D. THERAPEUTIC COMPOSITIONS AND METHODS

The present disclosure provides new methods of regenerating tissues. The tissues may be neuronal in origin, including spinal nerves, but can be applied to other types of tissues.

1. Pharmaceutical Formulations

It will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to materials stable and allow for uptake by target cells. Buffers also will be employed when compositions are introduced into a patient. Aqueous compositions of the present disclosure comprise an effective amount of the substance to cells, dissolved or dispersed in a pharmaceutically acceptable carrier or aqueous medium or cells themselves. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the liposomes or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Administration may be by orthotopic, intradermal, subcutaneous, intramuscular, intraperitoneal, or intravenous injection. Such compositions would normally be administered as pharmaceutically acceptable compositions, described supra. The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

2. Methods

Cationic magnetoliposomes (CMLs) may be synthesized with SPIONs encapsulated in the core and neurotrophic growth factors hosted within the lipid bilayer. The CMLs are used to magnetically label the NSCs. After cellular internalization, an external magnetic field will be applied to induce the cumulative dipole moment in the CML-labeled NSCs. The consequent magnetic interactions between NSCs cause spontaneous formation of highly aligned chain/column lattices. The principal axis of the lattices will conform to the magnetic flux lines, serving as a virtual guide to the NSC proliferation and colonization. Sustained release of the neurotrophic growth factors stored in the CMLs will be triggered and controlled by a RF electromagnetic field. If desired, MRI can be used to noninvasively track various cellular events using the SPION core as contrast agents. In the foregoing procedures, synthesis of CMLs and magnetic labeling of NSCs will be conducted in vitro. Aqueous suspension of the CML-labeled NSCs can be injected directly into the injured spinal cord. Aligned NSC chain/column lattices will be spontaneously self-assembled in vivo when the magnetic field is applied. Thus all three critical requirements of an ideal scaffold for spinal cord nerve repair, i.e., injectability, alignability and bioactivity, can be satisfied.

### E. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

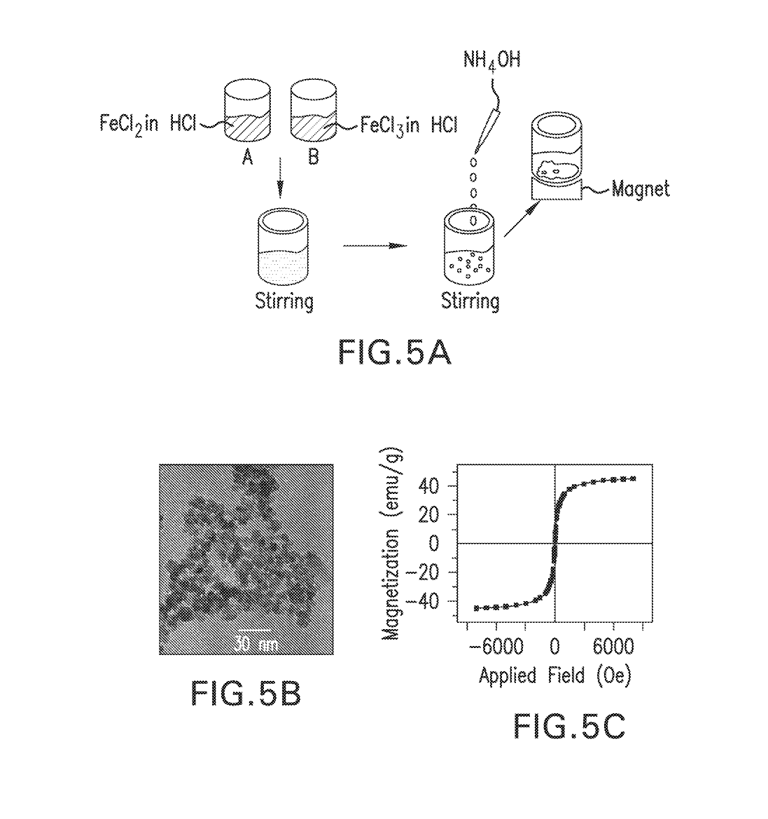

SPIONs have been synthesized via a co-precipitation process in the PIs' laboratory. As shown in FIG. 5A, iron oxides are formed from aqueous Fe²⁺/Fe³⁺ salt solutions by the addition of a base under inert atmosphere at room temperature. The size and composition of SPIONs are controlled by the type of salts used (e.g., chlorides, sulfates, nitrates), the Fe²⁺/Fe³⁺ ratio, the reaction temperature, the solution pH value and the ionic strength of the media. The size, composition and microstructure of the SPIONs have been characterized by X-ray diffraction (XRD) and transmission electron microscopy (TEM), as shown in FIG. 5B. Magnetic properties including the size dependent superparamagnetism and inter-particle interactions have been investigated using magnetic hysteresis and temperature-dependent field cool and zero-field magnetization measurements, as shown in FIG. 5C.

### Example 2

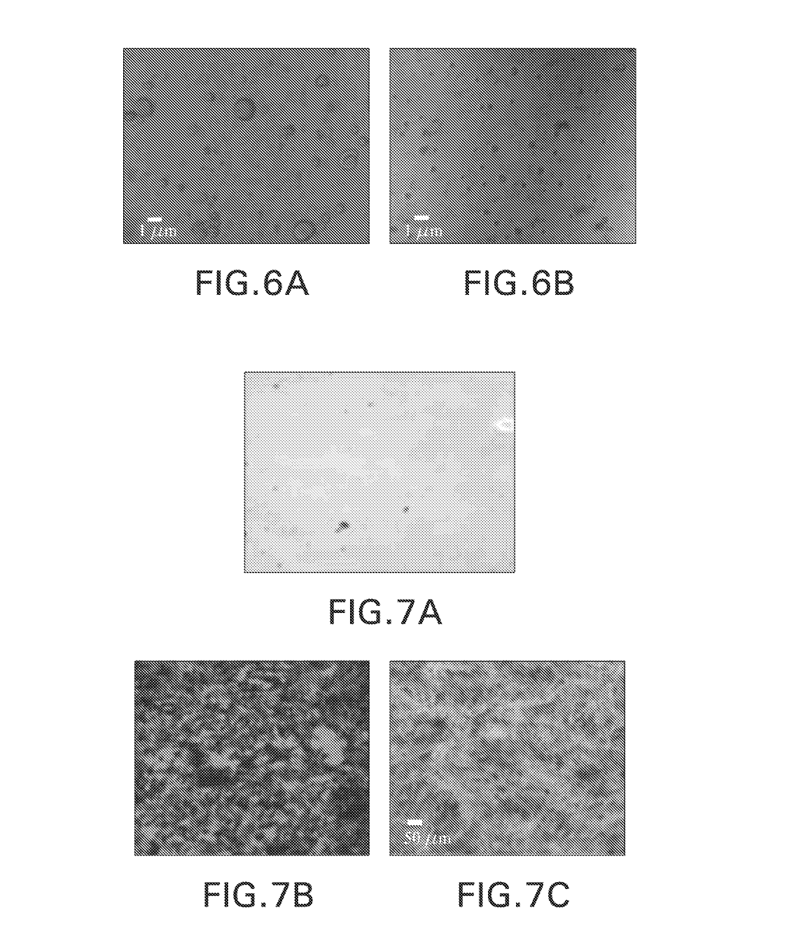

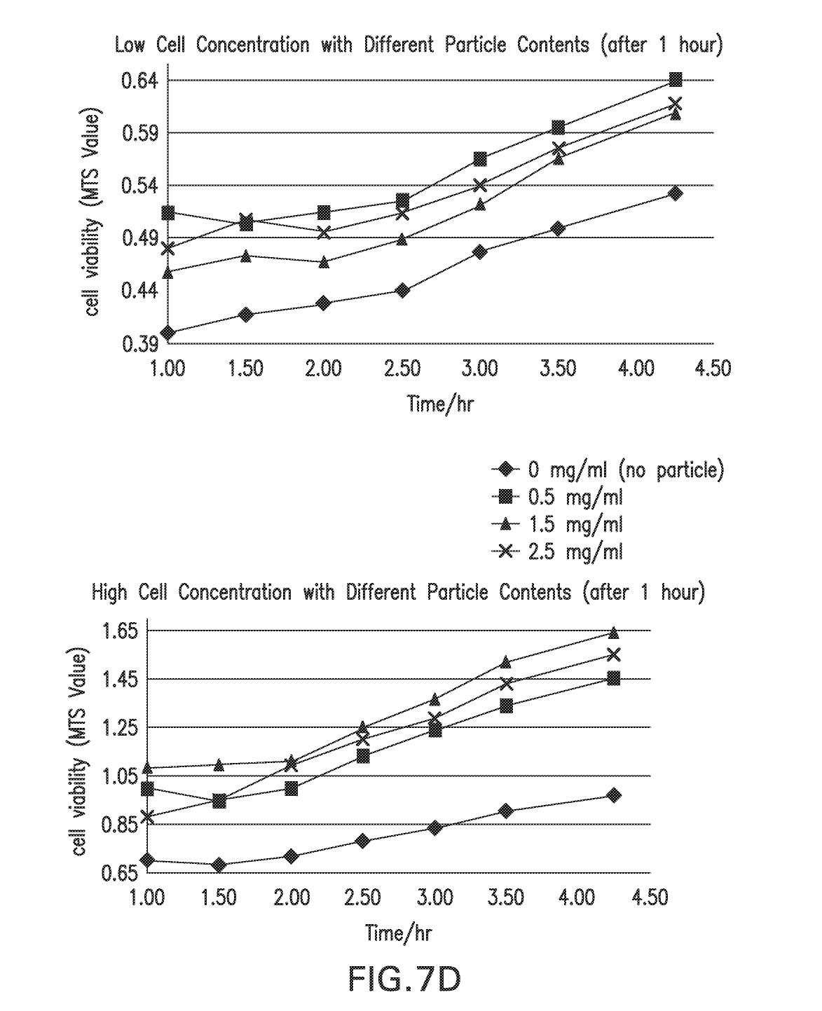

Liposomes can be produced using methods such as sonication, extrusion, homogenization, swelling, and electroformation [41-46]. A common deficiency of these techniques is the bilayer material of the liposomes is the same as what they enclose, thus limiting their utility for encapsulating drug molecules or other active agents. To identify a suitable liposomal synthesis route, the inverted emulsion [47] and reverse evaporation [48] methods have been explored. Cationic liposomes (CLs) without SPION-encapsulation were prepared using the reverse evaporation method. Briefly, the mixture of N-(α-trimethylammonioacetyl)-didodecyl-D-glutamate chloride (TMAG), dilauroylphosphatidylcholine (DLPC) and dioleoylphosphatidylethanolamine (DOPE) at a 1:2:2 molar ratio is dissolved in chloroform (CHCl₃). The excess solvent is removed by evaporation to yield a thin lipid film on the container wall surface. The CLs are formed after hydrating the lipid film with aqueous buffer and subsequent sonication. The microscopy image in FIG. 6A shows the CLs are polydispersed with a wide variation in the size range from 500 nm to several microns. Alternatively, CMLs are synthesized using inverted emulsion method, which involves the assembly of two independently formed monolayers of lipid. First, aqueous SPION solution is emulsified by mixing with the dodecane oil containing dissolved DOPE and DLPC. As the tiny drops of SPION solution are formed, the DOPE and DLPC molecules dispersed in the oil phase adsorb onto the drop surface to form a lipid monolayer, which will serve as the inner leaflet of the final bilayer structure. The size of surface-covered emulsion droplets determines the size of CMLs, and can be fine-tuned by ultrasonication and filtration through carbonate membrane with nanoscale pores. Subsequently, the emulsion droplets are mixed with a second oil phase, also containing dissolved DOPE and DLPC, and pick up a second layer of lipid on the surface, resulting in the bilayered CMLs. The micrograph in FIG. 7B shows the invert emulsion method yields smaller liposomes (generally less than 500 nm in diameter) with uniform size distribution. Thus it will be adopted for synthesizing the CMLs in the proposed work.

### Example 3

Biocompatibility of the synthesized SPIONs has been investigated using Rat-2 fibroblast cells as the model system. The Rat-2 fibroblast cells offer advantages including fast doubling time and relative ease of maintenance, and have been used to assess the toxicological response of several biomolecular systems, such as polypropene mesh, fibrinogen, and zirconia [49-51]. The cytotoxicity is studied using a combined approach of microscopic observation and Trypan blue viability staining. The results in FIGS. 7A-C show that bare SPIONs, SPIONs coated with N(CH₃)₄OH (tetramethyl-ammonium hydroxide for better dispersion) and SPIONs coated with dextran demonstrate very different biocompatibility.

### Example 4

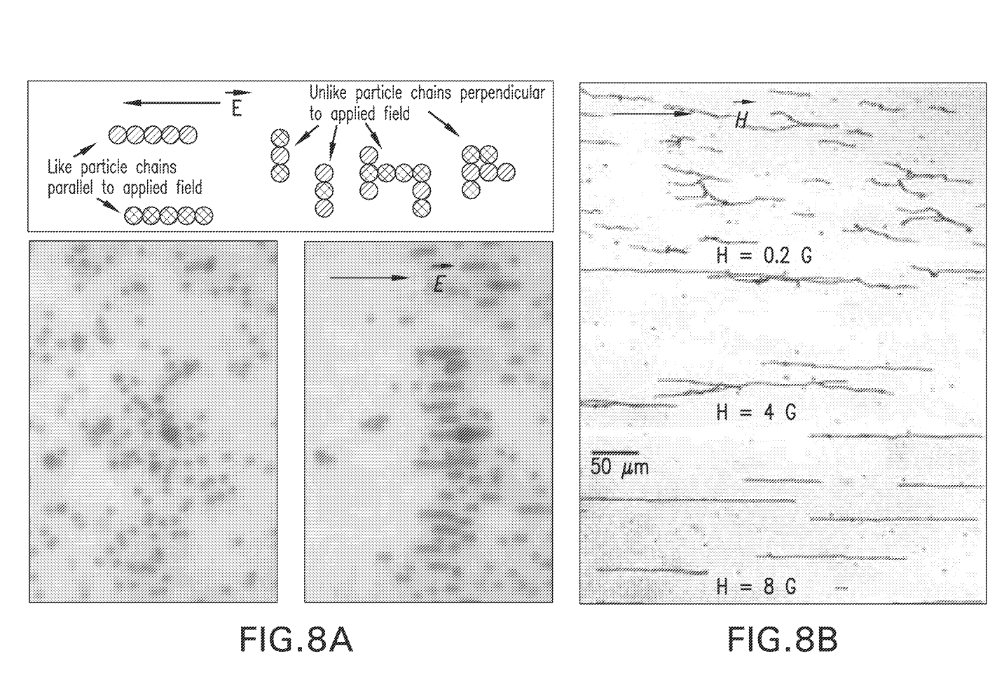

Colloidal solutions of micro/nanoparticles will spontaneously form highly-ordered 2D or 3D supraparticle architectures under controlled electric or magnetic field. Directed self-assembly of spherical and non-spherical particles has been investigated by the PIs, both experimentally and theoretically [52,53]. FIG. 8A illustrates that randomly dispersed polystyrene microparticles are self-assembled into chain lattices due to negative dielectrophoresis. FIG. 8B shows that free-standing magnetic nanowires in suspension can be aligned and grafted in a controlled magnetic field. In both cases, the principle axis of the self-assembled structure conforms to the direction of the field vector. Therefore, by controlling the direction, strength and frequency of the external electric or magnetic field, well-defined colloidal structures can be produced in the fluidic environment in situ using particles as the building block.

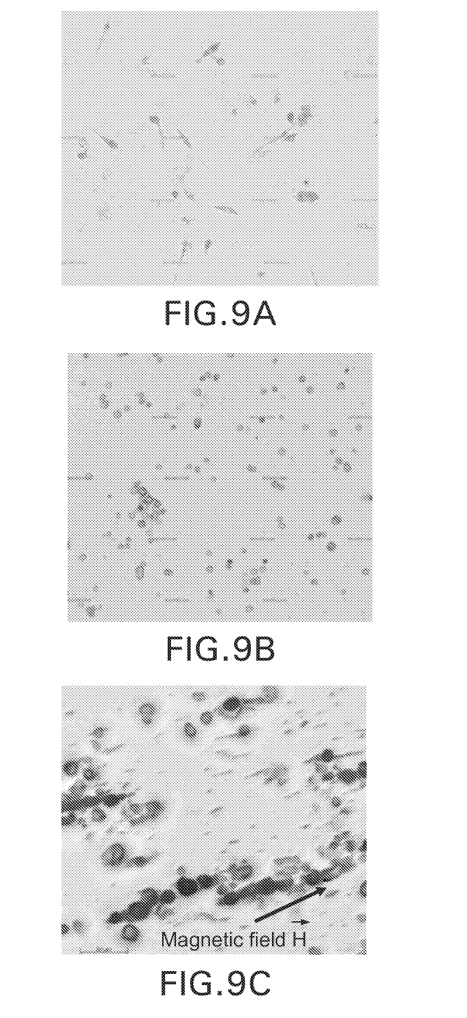

Magnetic directed self-assembly of oligodendrocyte precursor cells (OPCs) labelled with SPIONs has been explored in the preliminary work. As suggested by the biocompatibility study, the SPIONs are coated with dextran to ensure sufficient cellular viability after uptake. In the experiments, OPCs are seeded with dilute SPION solution and incubated for 24-48 hours in standard culture media at 37° C. and 5% CO₂. Before the self-assembly experiment, the cells are washed multiple times with phosphate-buffered saline to remove excess SPIONs and are then trypsinized. The slide carrying a drop of SPION-labeled OPCs is placed in a magnetic field created by two neodymium-iron-boron rare-earth magnets. The magnetic field strength is measured to be about 200 G. It is found self-assembly of OPCs occurs almost immediately. The optical microscopy images in FIGS. 9A-C show the sequential processes of cellular internalization, harvest and magnetic directed self-assembly.

### Example 5

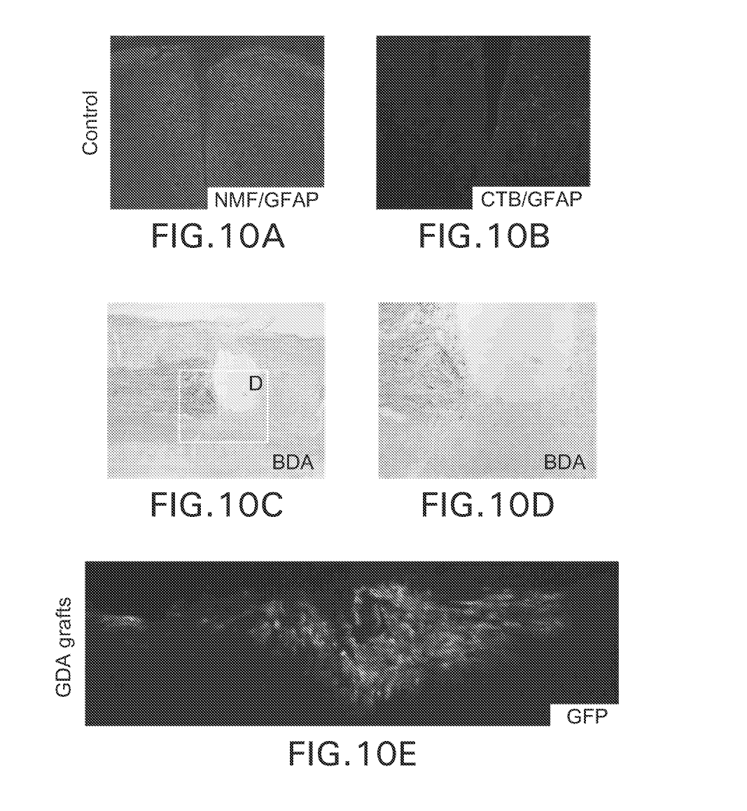

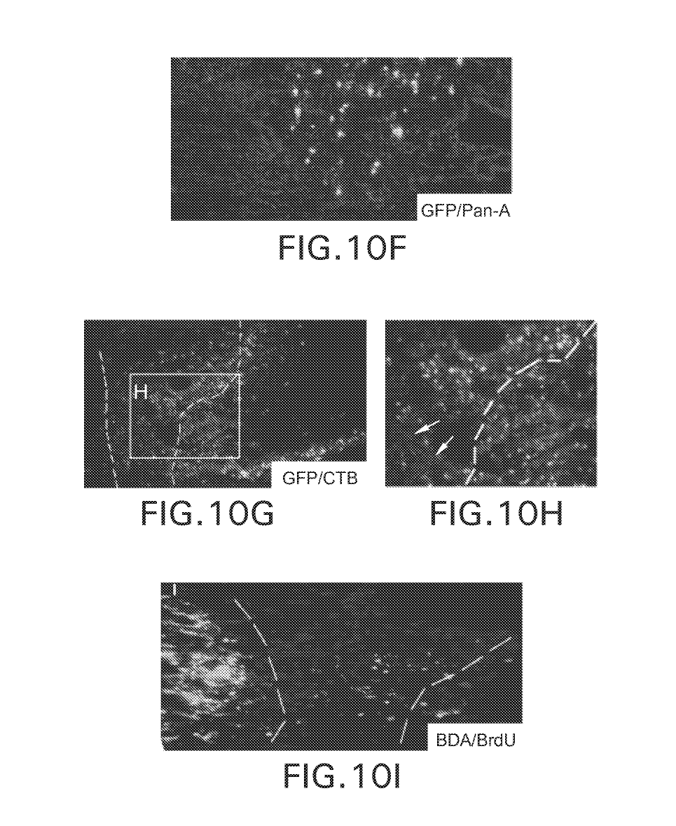

Results shown in FIGS. 10A-I demonstrate that transplanted NSCs can promote the regeneration of both ascending dorsal column (DC) axons and descending cortical-spinal-tract (CST) after SCI; however, the grafted NSCs are seen to be randomly oriented. Therefore, there is a critical need for a novel technology to highly align the grafted NSCs in the injured spinal cord longitudinally to further promote greater axonal regeneration.

### Example 6

Methods used to synthesize SPIONs will be optimized to gain better control of the particle size distribution, surface chemistry and magnetic properties. High resolution field-emission scanning electron microscopy (SEM) will be used to characterize the size and morphology of SPIONs. Since the formation of stable SPION colloids constitutes the first step of the proposed research, biocompatible organic substances, such as dextran, polyethylene glycol (PEG) or poly(ethylene oxide) (PEO), will be used to modify the surface of SPIONs to improve their stability in the solution phase. X-ray photoelectron spectroscopy (XPS) will be used to acquire bonding information of the surface coating if needed. Magnetic properties including hysteresis and temperature dependence will be measured by magnetometers, such as vector vibrating sample magnetometer (VSM) and superconducting quantum interference device (SQUID). Both intrinsic and extrinsic magnetic properties of the SPIONs before and after surface modification will be studied.

After formulating stable SPION colloids, CMLs will be produced using a modified invert emulsion method. It was found [38] that, although the efficiency of cellular uptake increases in proportion to the content of cationic lipid in the ML bilayer, severe toxic effect will arise if the cationic lipid level exceed a certain threshold value. Thus, in this work, SPION-free liposomes containing cationic lipids and neutral MLs are first synthesized separately. They are then incubated together. During incubation, the cationic lipid in the SPION-free liposomes will transfer spontaneously to the outer leaflet of the bilayer of MLs. By adjusting the time of incubation period, the amount of cationic lipid in the produced CMLs can be fine-tuned. The optimal synthesis conditions will be determined in order to produce CMLs for efficient cell labeling without harming the cell viability. The inclusion of neurotrophin factors, such as neurotrophin 3 (NT3) and brain derived neurotrophin factor (BDNF), will be achieved by the pH gradient method or the ammonium sulfate method [54, 55].

### Example 7

Magnetic labeling of NSCs will be achieved by incubating the mixture of CML solution and cultured NSCs for 24 hours under normal growth conditions. Since the cellular membranes bear an overall negative charge, it is hypothesized that positively-charged CMLs can be taken up easily by the cells via electrostatic interaction. In practice, however, the nature of the CML-cell association is crucial to effective magnetic labeling, i.e., whether CMLs merely adsorb to the external side of the cellular membrane or they are indeed internalized. In this project, the intracellular CML uptake will be visualized by optical microscopy with Prussian blue staining and by TEM. After drying the cell suspension and digesting it with perchloric and nitric acid, the iron content of the labeled NSCs will be assessed using an UV spectrophotometer and MR relaxometry [56, 57]. Since this technique only yields the mean population value of iron concentration in dead cells, a more accurate measurement of the SPION uptake in individual live cells will be conducted using the magnetophoresis-based cell tracking velocimetry method [34]. This method utilizes the principle of particle image velocimetry [58] to measure the cell magnetophoretic mobility, which can be directly related to the physicochemical properties of the cell-label complex. In brief, magnetically susceptible cells undergo translational motion in a viscous medium under the influence of an applied magnetic field [symbol] . The cell experiences a constant terminal velocity when the magnetic driving force is balanced by hydrodynamic viscous drag. The mass of iron in the cell (m) can be deduced from the measured cell magnetophoretic mobility:

(M̅M) as m=MM(3π·a·μ·B)/(μ₀Mₛ)

where μ₀ is the magnetic permeability in a vacuum, Mₛ the mass saturation magnetization of iron, a the cell diameter, and μ the viscosity of the fluid medium. This method also allows a rough estimate of the number of SPIONs that are phagocytized by the cell.

Toxicity of the CML-labeled NSCs will be studied to ensure the viability and normal proliferation and differentiation of the cells. A 3-(4,5-dimethylthiazol-2-yl)-2,5-diphenyl tetrazolium bromide (MTT) assay will be used for this purpose. Viable cells produce mitochondrial dehydrogenases, which will convert the yellow soluble MTT solution into blue-violet insoluble formazan crystals. Measurement of the absorbance of the formazan product at a wavelength of 570 nm will yield information of the cellular viability. Pulse chase experiments will be performed to determine the long-term cell ability to continue to proliferate. To investigate if labeled NSCs still possess multipotent differentiation, NSCs will be induced to differentiate by withdrawal of FGF2 for four days. The percentage of NSCs to differentiate into neurons, astrocytes and oligodendrocytes will be determined by immunohistochemistry using antibodies against MAP2, GFAP, and MBP, respectively. The toxicity study will be repeated for CMLs produced with different cationic lipid levels to establish the dose-response relationship. The information will be fed back to the CML synthesis process to determine the optimal CML composition for efficient cell labeling without harming the cell viability, proliferation and differentiation.

### Example 8

The efficiency of neural stem cell-based therapy for SCI repair depends critically on how well the regenerating NSCs can orient themselves along a preferential direction, i.e., longitudinally in the spinal cord, to re-establish the nerve connection. The proposed research utilizes the spontaneous self-assembly of paramagnetic particles, i.e., the CML-labeled NSCs, into aligned chain/column lattices to restore the directional information lost in SCI. Therefore, a fundamental understanding of the mechanisms and kinetics of the magnetic directed assembly process is needed for devising optimal conditions to ensure good alignment of the labeled NSCs, particular in vivo. Both theoretical and experimental approaches will be used for this purpose.

From the measurement of iron content in CML-labeled NSCs, an effective volume magnetic susceptibility λ_{eff} can be inferred for the NSCs. When an external magnetic field is applied, the NSCs acquire magnetic dipole moments. The induced dipole moment in an individual NSC is:

[symbol]

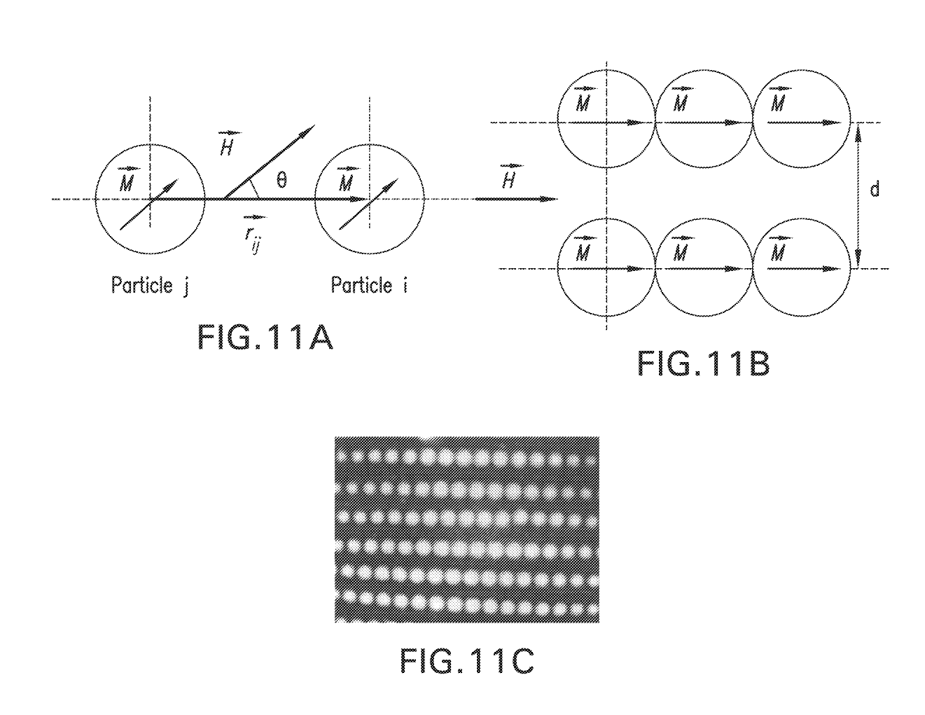

=(1/6)πa³λ_{eff} [symbol] (Eq. 1)

where a is the diameter of the NSC and [symbol] is the external field vector. The dipole-dipole interaction between two NSCs is readily described by an anisotropic interaction potential U_{d} and a repulsive potential barrier Uᵣ which prevents the cells from approaching each other [59, 60]:

$${U}_{d} = \frac{{M}^{2} {μ}_{0}}{4 π} ( \frac{1 - 3 {cos}^{2} θ}{{r}_{ij}^{3}} ) \\ {U}_{r} = \frac{{M}^{2}}{4 π {μ}_{0} a} exp [ - 40 ( \frac{{r}_{ij}}{a} ) ] \qquad ( Eq . 2 \text{-} 3 )$$

where, as shown in FIG. 11A, rᵢⱼ is the magnitude of the displacement vector [symbol] ᵢⱼ from cell j to cell i, and θ is the angle between [symbol] ᵢⱼ and the external field vector. If the dipole-dipole interactions are stronger than the thermal fluctuation due to Brownian motion, i.e., the parameter λ=μ₀M²/(16πa³k_{B}T)>>1 where k_{B} is the Boltzmann constant and T is the temperature, the particles will undergo head-to-tail aggregation and self-assemble into chain lattices aligned along the direction of the external field, as shown in FIG. 11B and (c), since this configuration (θ=0) leads to the minimum system energy [61-63].

Two neighboring cell chains may interact laterally and undergo secondary aggregation. The lateral interaction energy per unit length a is on the order of:

$$U \text{∼} \frac{{χ}_{eff} {H ( {μ}_{0} {k}_{B} T )}^{1 / 2} {a}^{5 / 2}}{{d}^{2}} \qquad ( Eq . 4 )$$

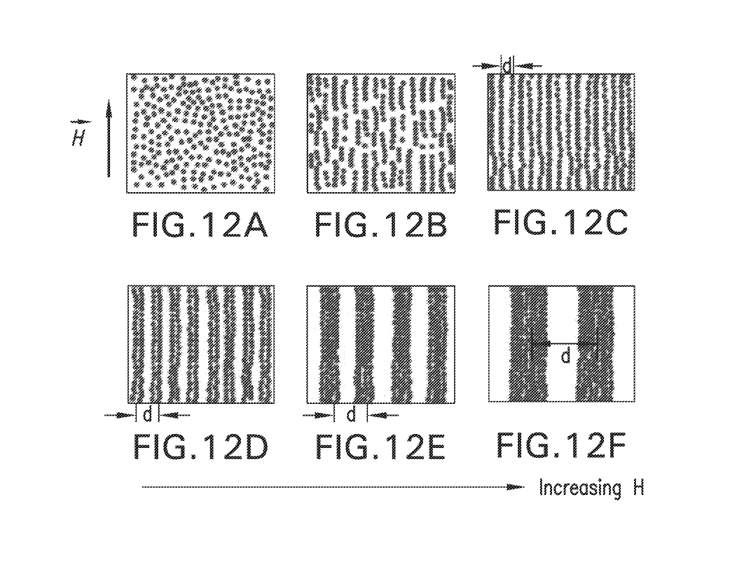

This energy strongly depends on the field strength H and the separation d between the chains (d is shown in both FIG. 11B and FIGS. 12A-B). As the field strength increases, the interaction energy also increases. When this energy becomes high enough to overcome the repulsive potential energy barrier, the two chains will coalesce laterally into a column. The corresponding structural transition can be seen from FIGS. 12C-D. Subsequently, the separation d between the newly formed columns would increase in order to decrease the overall energy of the system, as suggested by Eq. 4. As depicted in FIGS. 12D-F, this coalescence process repeats itself as the field strength increases to the next critical level, eventually giving rise to an equilibrium configuration which consists of equally spaced uniformly distributed chain columns. In addition to the field strength, the equilibrium configuration is also affected by the volume fraction of the particles as well as the maximum allowable span of the lattice [64-67]. Nevertheless, it is clear that the cellular pattern formed in FIG. 12F resembles the collateral never fibers in healthy spinal cords, which will be the ultimate pursuit of this project.

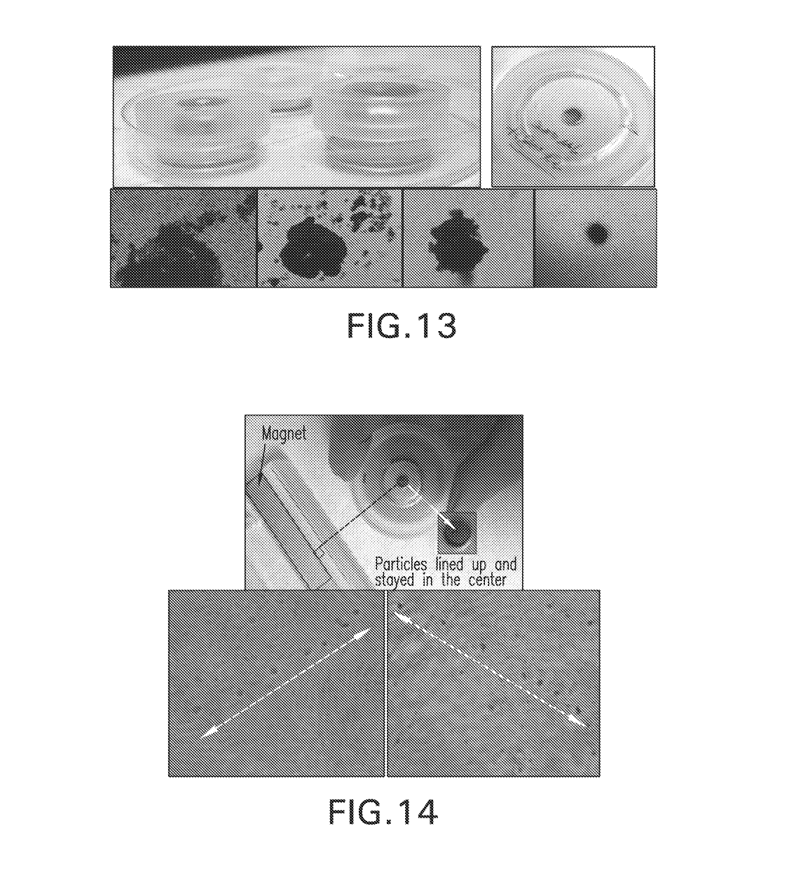

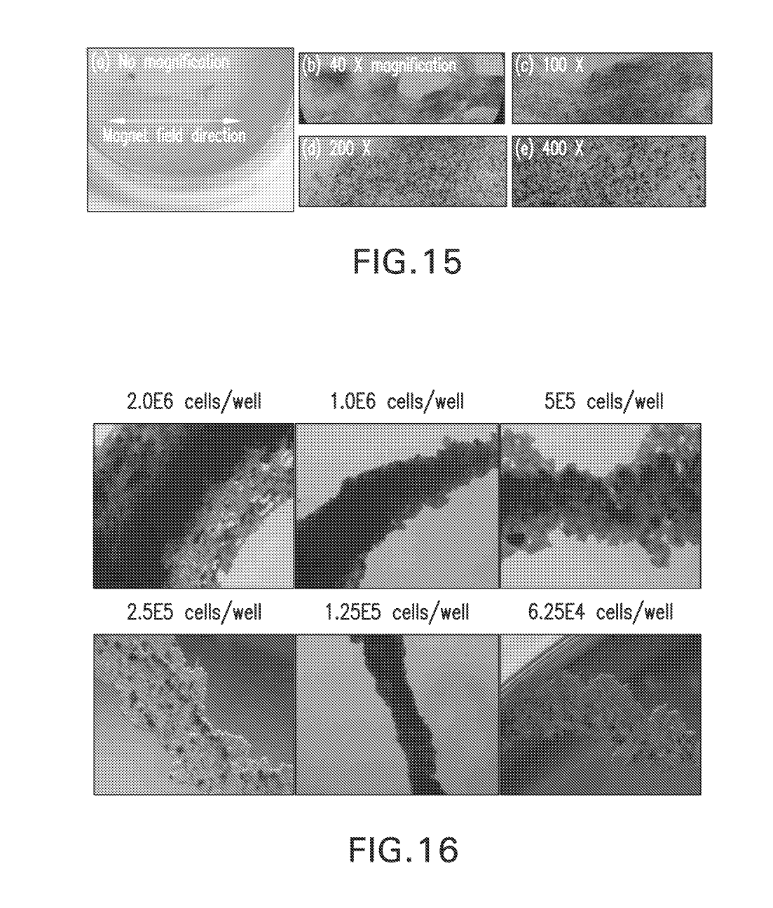

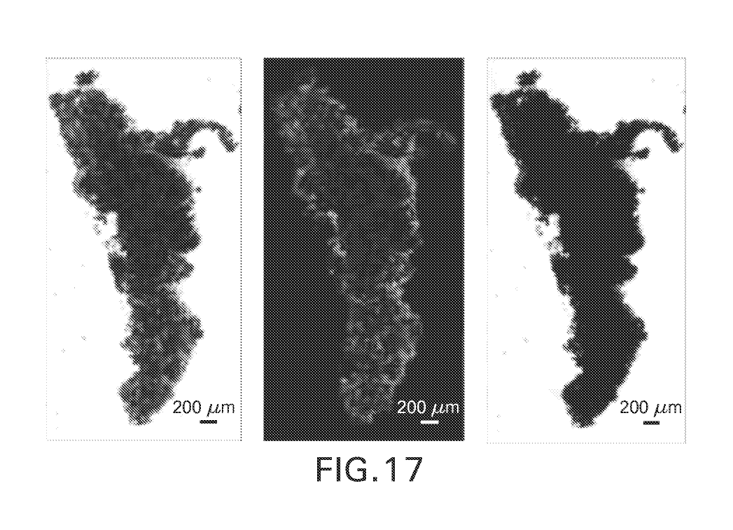

Theoretically, the Brownian dynamics simulation (BDS) method will be used to investigate the pattern formation kinetics and to establish the relation between the lattice structure formed and the control parameters. In the BDS method, the CML-labeled NSC will be considered as a composite particle that consists of a single magnetic domain core and a coating layer of surfactant molecules [68], as shown in FIG. 13B. The particle interaction potentials will be considered, including the dipole-dipole potential (Eq. 2), the repulsive potential between the SPION cores (Eq. 3), as well as the repulsive potential caused by the surfactant-surfactant interaction which is given by:

$${U}_{suy} = 2 π {RNk}_{B} T [ 2 - \frac{{r}_{ij} - 2 R}{δ} - \frac{{r}_{ij}}{δ} ln ( \frac{2 δ + 2 R}{{r}_{ij}} ) ] \qquad ( Eq . 5 )$$

where R, N and δ are the radius of the particle, the surface number density of the surfactant molecules and the thickness of the surfactant layer, respectively. Neglecting the inertial effect, the translational motion of the particles is described by the Langevin equation:

$$\vec{u} = \frac{\vec{d r}}{d t} = ( {\vec{F}}_{M} + {\vec{F}}_{B} ) / ( 6 π μ R ) \qquad ( Eq . 6 )$$

where [symbol] is the displace vector, [symbol] is the particle velocity, [symbol] _{M} are [symbol] _{B} the magnetic force and the random force due to Brownian motion, respectively; m is the mass of the particle, and μ is the viscosity of the fluid. The random force also satisfies:

[symbol]

[symbol]

_{B}(t) [symbol] =0

[symbol]

[symbol]

_{B}(t) [symbol] _{B}(t+Δt) [symbol] =12πμRk_{B}T·δ(t) (Eq. 7-8)

where δ(t) is the Dirac delta function. The simulation will be performed for a computation domain shown in FIG. 13B, where periodic boundary conditions are applied for x and y directions. The effects of various control parameters, including the field strength, effective magnetic susceptibility, volume fraction of the NSCs and maximum allowable span of the lattice, on the cellular pattern structure and the formation kinetics will be studied in details.

Experimentally, the self-assembly kinetics of CML-labeled NSCs will be studied with optical microscopy. The two primary goals are: 1) to determine the dependence of equilibrium lattice structure on control parameters, and 2) to characterize the time evolution of the mean length of cellular chains/columns formed. The measurements will be compared to the BDS results. Also, the asymptotic scaling law of the mean chain size <s(t)> with respect to time, <s(t)>˜t^{z}, where z is the scaling factor [69], will be critically assessed.

In the experiments, suspensions of CML-labeled NSCs will be stored in a microcapillary tube with a square cross section. The tube is placed horizontally in the center of a set of Helmholtz coils, which generates a uniform magnetic field throughout the sample. The field strength can be adjusted by tuning the current through the coils. Time evolution of the cellular chain/column size will be recorded with a high-speed camera under a microscope. A number of objective lenses will be used to achieve high magnification and a dynamic range of working distance. The digital images are then processed to yield the relevant statistics. The experiments are to be conducted for NSCs labeled with different loadings of SPIONs over a range of cellular volume concentrations and magnetic field strengths.

All of the compositions and/or methods disclosed and claimed herein can be made and executed without undue experimentation in light of the present disclosure. while the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
//neuroscience.stanford.edu/research/programs/program_info/Spine_pdf%20final_040.809.pdf
Kerschensteiner, M., Schwab, M. E., Lichtman, J. W., and Misgeld, T., 2005, “In vivo imaging of axonal degeneration and regeneration in the injured spinal cord,” Nature Medicine, 11(5), pp. 572-577.
Fawcett, J. W., 2006, “Overcoming inhibition in the damaged spinal cord,” Journal of Neurotrauma, 23(3-4), pp. 371-383.
Silver, J., and Miller, J. H., 2004, “Regeneration beyond the glial scar,” Nature Reviews Neuroscience, 5(2), pp. 146-156.
Neumann, S., Skinner, K., and Basbaum, A. I., 2005, “Sustaining intrinsic growth capacity of adult neurons promotes spinal cord regeneration,” P Natl Acad Sci USA, 102(46), pp. 16848-16852.
Neumann, S., Bradke, F., Tessier-Lavigne, M., and Basbaum, A. I., 2002, “Regeneration of sensory axons within the injured spinal cord induced by intraganglionic cAMP elevation,” Neuron, 34(6), pp. 885-893.
Qiu, J., Cai, C. M., Dai, H. N., McAtee, M., Hoffman, P. N., Bregman, B. S., and Filbin, M. T., 2002, “Spinal axon regeneration induced by elevation of cyclic AMP,” Neuron, 34(6), pp. 895-903.
Romero, M. I., Rangappa, N., Garry, M. G., and Smith, G. M., 2001, “Functional regeneration of chronically injured sensory afferents into adult spinal cord after neurotrophin gene therapy,” Journal of Neuroscience, 21(21), pp. 8408-8416.
Ramer, M. S., Priestley, J. V., and McMahon, S. B., 2000, “Functional regeneration of sensory axone into the adult spinal cord,” Nature, 403(6767), pp. 312-316.
Lu, P., Jones, L. L., Snyder, E. Y., and Tuszynski, M. H., 2003, “Neural stem cells constitutively secrete neurotrophic factors and promote extensive host axonal growth after spinal cord injury,” Experimental Neurology, 181(2), pp. 115-129.
Pallini, R., Vitiani, L. R., Bez, A., Casalbore, P., Facchiano, F., Gerevini, V. D., Falchetti, M. L., Fernandez, E., Maira, G., Peschle, C., and Parati, E., 2005, “Homologous transplantation of neural stem cells to the injured spinal cord of mice,” Neurosurgery, 57(5), pp. 1014-1024.
Fabrizio Gelain, S. P., Stefania Antonini, Carla Cunha, Matteo Donega, Joseph Lowery, Francesca Taraballi, Gabriella Cerri, Marcella Montagna, Fausto Baldissera, and Angelo Vescovi, 2011, “Transplantation of Nanostructured Composite Scaffolds Results in the Regeneration of Chronically Injured Spinal Cords,” Acs Nano, 5(1), p. 9.
Chen, G., Hu, Y. R., Wan, H., Xia, L., Li, J. H., Yang, F., Qu, X., Wang, S. G., and Wang, Z. C., 2010, “Functional recovery following traumatic spinal cord injury mediated by a unique polymer scaffold seeded with neural stem cells and Schwann cells,” Chinese Medical Journal, 123(17), pp. 2424-2431.
Teng, Y. D., Lavik, E. B., Qu, X. L., Park, K. I., Ourednik, J., Zurakowski, D., Langer, R., and Snyder, E. Y., 2002, “Functional recovery following traumatic spinal cord injury mediated by a unique polymer scaffold seeded with neural stem cells,” P Natl Acad Sci USA, 99(5), pp. 3024-3029.
Deumens, R., Koopmans, G. C., Honig, W. M. M., Maquet, V., Jerome, R., Steinbusch, H. W. M., and Joosten, E. A. J., 2006, “Chronically injured corticospinal axons do not cross large spinal lesion gaps after a multifactorial transplantation strategy using olfactory ensheathing cell/olfactory nerve fibroblast-biomatrix bridges,” Journal of Neuroscience Research, 83(5), pp. 811-820.
Petri-Fink, A., and Hofmann, H., 2007, “Superparamagnetic iron oxide nanoparticles (SPIONs): From synthesis to in vivo studies—A summary of the synthesis, characterization, in vitro, and in vivo investigations of SPIONs with particular focus on surface and colloidal properties,” Ieee Transactions on Nanobioscience, 6(4), pp. 289-297.
Dobson, J., 2008, “Remote control of cellular behaviour with magnetic nanoparticles,” Nature Nanotechnology, 3(3), pp. 139-143.
Lin, M. M., Kim, D. K., El Haj, A. J., and Dobson, J., 2008, “Development of Superparamagnetic Iron Oxide Nanoparticles (SPIONS) for Translation to Clinical Applications,” Ieee Transactions on Nanobioscience, 7(4), pp. 298-305.
Rosengart, A. J., Kaminski, M. D., Chen, H. T., Caviness, P. L., Ebner, A. D., and Ritter, J. A., 2005, “Magnetizable implants and functionalized magnetic carriers: A novel approach for noninvasive yet targeted drug delivery,” Journal of Magnetism and Magnetic Materials, 293(1), pp. 633-638.
Gneveckow, U., Jordan, A., Scholz, R., Bruss, V., Waldofner, N., Ricke, J., Feussner, A., Hildebrandt, B., Rau, B., and Wust, P., 2004, “Description and characterization of the novel hyperthermia- and thermoablation-system MFH® 300F for clinical magnetic fluid hyperthermia,” Medical Physics, 31(6), pp. 1444-1451.
Sigal, R., Vogl, T., Casselman, J., Moulin, G., Veillon, F., Hermans, R., Dubrulle, F., Viala, J., Bosq, J., Mack, M., Depondt, M., Mattelaer, C., Petit, P., Champsaur, P., Reihm, S., Dadashitazehozi, Y., de Jaegere, T., Marchal, G., Chevalier, D., Lemaitre, L., Kubiak, C., Helmberger, R., and Halimi, P., 2002, “Lymph node metastases from head and neck squamous cell carcinoma: MR imaging with ultrasmall superparamagnetic iron oxide particles (Sinerem MR)—results of a phase-III multicenter clinical trial,” European Radiology, 12(5), pp. 1104-1113.
H. Kalish, A. S. A., B. R. Miller, B. K. Lewis, H. A. Zywicke, J. W. M. Bulte, L. H. Bryant Jr., J. A. Frank, 2003, “Combination of Transfection Agents and Magnetic Resonance Contrast Agents for Cellular Imaging: Relationship Between Relaxivities, Electrostatic Forces, and Chemical Composition,” Magnetic Resonance in Medicine, 50(2), p. 8.
Hughes, S., Dobson, J., and El Haj, A. J., 2007, “Magnetic targeting of mechanosensors in bone cells for tissue engineering applications,” Journal of Biomechanics, 40, pp. S96-S104.
Hughes, S., El Haj, A. J., and Dobson, J., 2005, “Magnetic micro- and nanoparticle mediated activation of mechanosensitive ion channels,” Medical Engineering & Physics, 27(9), pp. 754-762.
Hughes, S., McBain, S., Dobson, J., and El Haj, A. J., 2008, “Selective activation of mechanosensitive ion channels using magnetic particles,” Journal of the Royal Society Interface, 5(25), pp. 855-863.
Melle, S., Rubio, M. A., and Fuller, G. G., 2001, “Time scaling regimes in aggregation of magnetic dipolar particles: Scattering dichroism results,” Physical Review Letters, 87(11), pp. art. no.-115501.
Skjeltorp, A. T., 1985, “ORDERING PHENOMENA OF PARTICLES DISPERSED IN MAGNETIC FLUIDS,” J Appl Phys, 57(8), pp. 3285-3290.
Wen, W. J., Zhang, L. Y., and Sheng, P., 2000, “Planar magnetic colloidal crystals,” Physical Review Letters, 85(25), pp. 5464-5467.
Alsberg, E., Feinstein, E., Joy, M. P., Prentiss, M., and Ingber, D. E., 2006, “Magnetically-guided self-assembly of fibrin matrices with ordered nano-scale structure for tissue engineering,” Tissue Engineering, 12(11), pp. 3247-3256.
Ino, K., Ito, A., and Honda, H., 2007, “Cell patterning using magnetite nanoparticles and magnetic force,” Biotechnology and Bioengineering, 97(5), pp. 1309-1317.
Ino, K., Ito, A., Kumazawa, H., Kagami, H., Ueda, M., and Honda, H., 2007, “Incorporation of capillary-like structures into dermal cell sheets constructed by magnetic force-based tissue engineering,” Journal of Chemical Engineering of Japan, 40(1), pp. 51-58.
Ito, A., Ino, K., Hayashida, M., Kobayashi, T., Matsunuma, H., Kagami, H., Ueda, M., and Honda, H., 2005, “Novel methodology for fabrication of tissue-engineered tubular constructs using magnetite nanoparticles and magnetic force,” Tissue Engineering, 11(9-10), pp. 1553-1561.
Shimizu, K., Ito, A., Lee, J. K., Yoshida, T., Miwa, K., Ishiguro, H., Numaguchi, Y., Murohara, T., Kodama, I., and Honda, H., 2007, “Construction of multi-layered cardiomyocyte sheets using magnetite nanoparticles and magnetic force,” Biotechnology and Bioengineering, 96(4), pp. 803-809.
Jing, Y., Mal, N., Williams, P. S., Mayorga, M., Penn, M. S., Chalmers, J. J., and Zborowski, M., 2008, “Quantitative intracellular magnetic nanoparticle uptake measured by live cell magnetophoresis,” Faseb Journal, 22(12), pp. 4239-4247.
Bulte, J. W. M., Laughlin, P. G., Jordan, E. K., Tran, V. A., Vymazal, J., and Frank, J. A., 1996, “Tagging of T cells with superparamagnetic iron oxide: Uptake kinetics and relaxometry,” Academic Radiology, 3, pp. S301-S303.
Sipe, J. C., Filippi, M., Martino, G., Furlan, R., Rocca, M. A., Rovaris, M., Bergami, A., Zyroff, J., Scotti, G., and Comi, G., 1999, “Method for intracellular magnetic labeling of human mononuclear cells using approved iron contrast agents,” Magnetic Resonance Imaging, 17(10), pp. 1521-1523.
Soenen, S. J. H., Hodenius, M., and De Cuyper, M., 2009, “Magnetoliposomes: versatile innovative nanocolloids for use in biotechnology and biomedicine,” Nanomedicine, 4(2), pp. 177-191.
Soenen, S. J. H., Baert, J., and De Cuyper, M., 2007, “Optimal conditions for labelling of 3T3 fibroblasts with magnetoliposomes without affecting cellular viability,” Chembiochem, 8(17), pp. 2067-2077.
De Cuyper M, J. M., 1988, “Magnetoliposomes: Formation and structural characterization,” european biophysics journal, 15(3), p. 8.
Chen, Y. J., Bose, A., and Bothun, G. D., 2010, “Controlled Release from Bilayer-Decorated Magnetoliposomes via Electromagnetic Heating,” Acs Nano, 4(6), pp. 3215-3221.
Waterhouse, D. N., Madden, T. D., Cullis, P. R., Bally, M. B., Mayer, L. D., and Webb, M. S., 2005, “Preparation, characterization, and biological analysis of liposomal formulations of vincristine,” Liposomes, Pt E, pp. 40-57.
Candiani, G., Pezzoli, D., Ciani, L., Chiesa, R., and Ristori, S., 2010, “Bioreducible Liposomes for Gene Delivery: From the Formulation to the Mechanism of Action,” Plos One, 5(10).
Negishi, Y., Endo-Takahashi, Y., Suzuki, R., Maruyama, K., and Aramaki, Y., 2010, “Development of Gene Delivery System into Skeletal Muscles by Bubble Liposomes and Ultrasound,” Yakugaku Zasshi-Journal of the Pharmaceutical Society of Japan, 130(11), pp. 1489-1496.
Pezzoli, D., Ciani, L., Ristori, S., Cigada, A., Chiesa, R., and Candiani, G., 2010, “Formulation of new reducible liposomes for gene delivery,” Drug Discovery Today, 15(23-24), p. A60.
Ito, H., Nonogaki, Y., Kato, R., and Honda, H., 2010, “Practical cell labeling with magnetite cationic liposomes for cell manipulation,” Journal of Bioscience and Bioengineering, 110(1), pp. 124-129.
Yanase, M., Shinkai, M., Honda, H., Wakabayashi, T., Yoshida, J., and Kobayashi, T., 1998, “Intracellular hyperthermia for cancer using magnetite cationic liposomes: An in vivo study,” Japanese Journal of Cancer Research, 89(4), pp. 463-469.
Pautot, S., Frisken, B. J., and Weitz, D. A., 2003, “Production of unilamellar vesicles using an inverted emulsion,” Langmuir, 19(7), pp. 2870-2879.
Szoka, F., and Papahadjopoulos, D., 1978, “PROCEDURE FOR PREPARATION OF LIPOSOMES WITH LARGE INTERNAL AQUEOUS SPACE AND HIGH CAPTURE BY REVERSE-PHASE EVAPORATION,” P Natl Acad Sci USA, 75(9), pp. 4194-4198.
Weyhe, D., Hoffmann, P., Belyaev, O., Mros, K., Muller, C., Uhl, W., and Schmitz, F., 2007, “The role of TGF-beta 1 as a determinant of foreign body reaction to alloplastic materials in rat fibroblast cultures: comparison of different commercially available polypropylene meshes for hernia repair,” Regulatory Peptides, 138(1), pp. 10-14.
Raffaelli, L., Iommetti, P. R., Piccioni, E., Toesca, A., Serini, S., Resci, F., Missori, M., De Spirito, M., Manicone, P. F., and Calviello, G., 2008, “Growth, viability, adhesion potential, and fibronectin expression in fibroblasts cultured on zirconia or feldspatic ceramics in vitro,” Journal of Biomedical Materials Research Part A, 86A(4), pp. 959-968.
Viney, M. E., Bullock, A. J., Day, M. J., and MacNeil, S., 2009, “Co-culture of intestinal epithelial and stromal cells in 3D collagen-based environments,” Regenerative Medicine, 4(3), pp. 397-406.
Liu, D., and Garimella, S. V., 2009, “Microfluidic Pumping Based on Traveling-Wave Dielectrophoresis,” Nanoscale and Microscale Thermophysical Engineering, 13(2), pp. 109-133.
Tanase, M., Bauer, L. A., Hultgren, A., Silevitch, D. M., Sun, L., Reich, D. H., Searson, P. C., and Meyer, G. J., 2001, “Magnetic alignment of fluorescent nanowires,” Nano Letters, 1(3), pp. 155-158.
Haran, G., Cohen, R., Bar, L. K., and Barenholz, Y., 1993, “TRANSMEMBRANE AMMONIUM-SULFATE GRADIENTS IN LIPOSOMES PRODUCE EFFICIENT AND STABLE ENTRAPMENT OF AMPHIPATHIC WEAK BASES,” Biochimica Et Biophysica Acta, 1151(2), pp. 201-215.
Haran, G., Cohen, R., Bar, L. K., and Barenholz, Y., 1994, “TRANSMEMBRANE AMMONIUM-SULFATE GRADIENTS IN LIPOSOMES PRODUCE EFFICIENT AND STABLE ENTRAPMENT OF AMPHIPATHIC WEAK BASES (VOL 1151, PG 201, 1993),” Biochimica Et Biophysica Acta-Biomembranes, 1190(1), pp. 197-197.
Rad, A. M., Janic, B., Iskander, A., Soltanian-Zadeh, H., and Arbab, A. S., 2007, “Measurement of quantity of iron in magnetically labeled cells: comparison among different UV/VIS spectrometric methods,” Biotechniques, 43(5), pp. 627-+.
Frank, J. A., Miller, B. R., Arbab, A. S., Zywicke, H. A., Jordan, E. K., Lewis, B. K., Bryant, L. H., and Bulte, J. W. M., 2003, “Clinically applicable labeling of mammalian and stem cells by combining; Superparamagnetic iron oxides and transfection agents,” Radiology, 228(2), pp. 480-487.
Liu, D., Garimella, S. V., and Wereley, S. T., 2005, “Infrared micro-particle image velocimetry in silicon-based microdevices,” Experiments in Fluids, 38(3), pp. 385-392.
Ukai, T., and Maekawa, T., 2004, “Patterns formed by paramagnetic particles in a horizontal layer of a magnetorheological fluid subjected to a dc magnetic field,” Physical Review E, 69(3).
Laskar, J. M., Philip, J., and Raj, B., 2010, “Experimental investigation of magnetic-field-induced aggregation kinetics in nonaqueous ferrofluids,” Physical Review E, 82(2).
Degennes, P. G., and Pincus, P. A., 1970, “PAIR CORRELATIONS IN A FERROMAGNETIC COLLOID,” Physik Der Kondensiterten Materie, 11(3), pp. 189-+.
Fermigier, M., and Gast, A. P., 1991, “AGGREGATION OF MAGNETIZABLE COLLOIDS,” Abstr Pap Am Chem S, 202, pp. 161-COLL.
Gross, M., and Kiskamp, S., 1997, “New long-range interaction between dipolar chains,” Physical Review Letters, 79(13), pp. 2566-2569.
Flores, G. A., Liu, J., Mohebi, M., and Jamasbi, N., 1999, “Magnetic field-induced nonequilibrium structures in a ferrofluid emulsion,” Physical Review E, 59(1), pp. 751-762.
Mohebi, M., Jamasbi, N., and Liu, J., 1996, “Simulation of the formation of nonequilibrium structures in magnetorheological fluids subject to an external magnetic field,” Physical Review E, 54(5), pp. 5407-5413.
Gulley, G. L., and Tao, R., 1993, “STATIC SHEAR-STRESS OF ELECTRORHEOLOGICAL FLUIDS,” Physical Review E, 48(4), pp. 2744-2751.
Gulley, G. L., and Tao, R., 1997, “Structures of an electrorheological fluid,” Physical Review E, 56(4), pp. 4328-4336.
Ukai, T., Maekawa, T., and Morimoto, H., 2001, “Brownian dynamics analysis of magnetic order-disorder transition in a ferromagnetic colloidal system,” International Journal of Modern Physics B, 15(6-7), pp. 817-822.
Promislow, J. H. E., Gast, A. P., and Fermigier, M., 1995, “AGGREGATION KINETICS OF PARAMAGNETIC COLLOIDAL PARTICLES,” Journal of Chemical Physics, 102(13), pp. 5492-5498.
Shields, L. B. E., Zhang, Y. P., Burke, D. A., Gray, R., and Shields, C. B., 2008, “Benefit of chondroitinase ABC on sensory axon regeneration in a laceration model of spinal cord injury in the rat,” Surgical Neurology, 69(6), pp. 568-577.

1. A method of making a cell comprising contacting said cell with a liposome comprising a plurality of magnetic iron particles.

2. The method of claim 1, wherein said cell is a stem cell.

3. The method of claim 1, wherein said liposome is a cationic liposome.

4. (canceled)

5. The method of claim 2, wherein the stem cell is a neural stem cell.

6. The method of claim 5, wherein said liposome further comprises a growth factor.

7. (canceled)

8. The method of claim 1, wherein said magnetic particles are iron oxide nanoparticles.

9. (canceled)

10. The method of claim 1, wherein said liposome is about 100 to about 500 nm in diameter.

11. The method of claim 1, wherein said iron magnetic particles are surface modified with a biocompatible organic molecule.

12. The method of claim 1, wherein said biocompatible organic molecule is dextran or a protein.

13. (canceled)

14. The method of claim 1, wherein said cell is a fibroblast.

15. A liposome comprising (a) a plurality of magnetic iron particles coated with a biocompatible organic polymer and (b) a growth factor.

16. The liposome of claim 15, wherein said growth factor is a neurogenic growth factor.

17. The liposome of claim 15, wherein said liposome is a cationic liposome.

18. (canceled)

19. A method of forming a cell structure comprising applying a stationary magnetic field to a plurality of cells comprising magnetic iron particles.

20. The method of claim 19, wherein the cell structure is a cell lattice or chain.

21. The method of claim 19, wherein said cell structure is formed in a living subject.

22. The method of claim 21, wherein said cell structure comprises neural stem cells.

23. The method of claim 22, wherein said cell structure is located in a nerve site in said subject.

24. The method of claim 23, wherein said subject suffers from a nerve deficit.

25. The method of claim 24, wherein said nerve deficit is a result of traumatic nerve injury, spinal cord injury or peripheral nerve injury.

26-27. (canceled)

28. The method of claim 24, wherein said nerve deficit is a result of a neurodegenerative disease.

29. (canceled)

30. The method of claim 19, wherein said liposome is a cationic liposome.

31. (canceled)

32. The method of claim 19, wherein said liposome further comprises a growth factor.

33. (canceled)

34. The method of claim 19, wherein said iron magnetic particles are oxide nanoparticles.

35. (canceled)

36. The method of claim 19, wherein said liposome is about 100 to about 500 nm in diameter.

37. The method of claim 19, wherein said iron magnetics particles are surface modified with a biocompatible organic molecule.

38. The method of claim 37, wherein said biocompatible organic polymer is dextran or a protein.

39. (canceled)

40. The method of claim 19, wherein said cell structure is formed from fibroblasts.
